(19) United States
(12) Patent Application Publication
(10) Pub. No.: US 2009/0221022 A1
(43) Pub. Date: Sep. 3, 2009

(54) Three Dimensional Cell Culture

(75) Inventors: Hilary Anne MacQueen, Bedfordshire (GB); Alison Jane Loughlin, Buckinghamshire (GB); Sandeep Daya, Buckinghamshire (GB)

Correspondence Address: WOLF GREENFIELD & SACKS, P.C., 600 ATLANTIC AVENUE, BOSTON, MA, 02210-2206, US

(73) Assignee: THE OPEN UNIVERSITY, Milton Keynes (GB)

(21) Appl. No.: 12/225,994

(22) PCT Filed: Mar. 28, 2007
(86) PCT No.: PCT/GB2007/050161

§ 371 Date: Apr. 3, 2009

(30) Foreign Application Priority Data
Apr. 5, 2006 (GB) 0606764.9

(51) Int. Cl. C12Q 1/02; C12N 5/02

(52) U.S. Cl. 435/29; 435/395; 435/377

(57) ABSTRACT

A method for culturing preadipocytes isolated ex vivo is described, the method including introducing preadipocytes into a three dimensional support matrix, and allowing the cells to differentiate in vitro into adipocytes within the support matrix. The matrix may be a collagen matrix. The method may be used for investigating the development of stem cells, or for investigating the response of adipocytes to stimuli. The method provides a system whereby adipocytes with biological properties resembling those in vivo can be grown in vitro.

### FIELD OF THE INVENTION

The present invention relates to a three-dimensional cell culture system for culturing adipocytes. The invention also relates to methods for culturing adipocytes and for obtaining adipocytes from preadipocytes in cell culture. Aspects of the invention further relate to methods for evaluating interactions between adipocytes and other cell types, and for evaluating responses of adipocytes to environmental stimuli. This enables a greater understanding of the mechanisms involved in cell and tissue interactions and has implications in the fields of obesity, diabetes, inflammation and related diseases.

### BACKGROUND TO THE INVENTION

Traditionally, adipose tissue has largely been considered as storage material that is otherwise inert. Its functional importance has only come to the fore over recent years. It is now known that adipocytes are involved in a range of interactions with other cell types, and in the production and secretion of various factors. Adipose tissue is associated with various tissues, including the heart, pericardium, perivascular and perirenal tissues, and bone marrow. It is also associated with lymphoid tissue, and adipocytes are known to interact with lymphoid cells. They secrete a variety of cytokines and chemokines, and are thought to be involved in the regulation of weight, as well as playing a role in diabetes and inflammation.

Adipocytes are large, fragile cells, and are difficult to maintain intact during analysis. If they rupture they release their fat contents, which can interfere with water-based biochemical assays. Considerable expertise is needed in adipocyte handling to minimise disruption.

Earlier work to culture adipocytes has focused on either explant cultures of tissue pieces or creating immortalised cell lines. There are drawbacks to both methods: explants quickly become necrotic, limiting the time over which analysis can be carried out, and cell lines lose the structural and functional properties of in vivo adipocytes. Adipocytes have also been isolated ex vivo and grown as monolayers. Again, as these cells proliferate in two dimensions they lose their ‘normal’ structure and function, and furthermore as adipocytes become fully mature (containing a large fat droplet) their density is such that they float off the monolayer and become lost, hindering analysis. The low buoyant density means that they are not amenable to conventional processes such as centrifugation. More recently, there has been a report of a three-dimensional culture system in which mature ex vivo adipocytes were co-cultured with endothelial cells in a collagen gel (Aoki et al, 2003, Cell Struct Funct 28, 55). This paper does not report the longevity of their cultures, nor the suitability of the cultured adipocytes for biochemical analysis. Moreover the co-cultures contained varying numbers of preadipocytes, which the authors considered a disadvantage. Further, Aoki et al report that the presence of endothelial cells in the culture is considered essential for growth of the adipocytes, and for such cells to develop from the preadipocytes.

WO 2005/121316 describes the culture of adipocytes or preadipocytes on the surface of a support matrix; effectively the matrix is acting as a two-dimensional culture substrate. There is no disclosure of differentiation of preadipocytes into adipocytes within the matrix.

WO 2004/035102 describes the culture of preadipocytes in particles having a fine bubble structure, and injecting these particles into the body. There is no disclosure of differentiation of the preadipocytes into adipocytes, and no mention of culture in vitro within a three-dimensional support matrix.

Hemmrich et al, Differentiation, Vol 73, 2005, pp 28-35, describe the in vitro differentiation of preadipocytes in a two-dimensional culture system.

Patel et al, Tissue Engineer, Vol 11, 2005, pp 1498-1505, describe the culture of preadipocytes in a cell scaffold system. No differentiation into adipocytes was seen, but only either cell death or cell proliferation.

Hilliou et al, Exp Cell Res, Vol 177, 1988, pp 372-381, discuss the culture of 3T3 preadipocyte cell lines.

Von Heimburg et al, Biomaterials, Vol 22, 2001, pp 429-438, describe the culture of preadipocytes in a collagen sponge produced by directional solidification and freeze-drying of collagen. There is no disclosure in this publication of the culture and differentiation of preadipocytes to adipocytes in vitro within a support matrix.

Kuberka et al, Int J Artificial Organs, Vol 25, 2002, pp 67-73, describe the creation of a collagen sponge similar to that used by von Heimburg, but do not describe culture of preadipocytes within the sponge.

Thus, none of the art discloses the culture of ex vivo preadipocytes within a three-dimensional support matrix, and their subsequent differentiation into adipocytes in vitro. The present inventors have developed a novel culture system and method intended to circumvent the prior art problems, at least in part. We provide a system in which adipocytes with biological properties resembling those in vivo can be grown in vitro in a stable substrate that renders them amenable to cytological, biochemical and molecular analysis. The culture system can be made to mimic closely the internal environment. This allows interactions between adipocytes and other cell types to be studied outside the body in a way that has not been possible before. For example, this may have applications in stem cell research. Stem cells in the body often reside and proliferate in microenvironments that include adipocytes. However, because they are so difficult to culture conventionally, adipocytes are frequently excluded from stem cell culture systems. Our 3D model could be used to provide adipocytes and other cells with an environment more closely resembling the in vivo one, and thereby offer better conditions for stem cell proliferation and differentiation.

### SUMMARY OF THE INVENTION

According to a first aspect of the present invention, there is provided a method of culturing adipocytes in vitro, the method comprising
introducing preadipocytes into a three dimensional support matrix; and
allowing the preadipocytes to differentiate into adipocytes within the support matrix.

The present method thus allows adipocytes to be grown within a matrix such that problems in handling and manipulating fragile adipocytes are reduced. Further, the three dimensional matrix retains mature adipocytes within the matrix, such that the mature cells do not float off a monolayer. The three dimensional matrix also provides an environment for the adipocytes which more closely resembles their natural environment, such that the form and function of the cells more closely resembles ‘normal’ cells.

The matrix preferably comprises a collagen matrix. Type I collagen is preferred, and may be provided in the form of gelatin (denatured type I collagen). Alternatively, type II collagen may be used. Type I collagen is the most preferred material, although we believe that other materials may instead be used. However, these may give less satisfactory results than the use of collagen. The purpose of the matrix is to provide a three dimensional support matrix for the cultured cells which permits migration of smaller cells, but which prevents migration of the larger mature adipocytes, while also permitting diffusion of culture medium, growth factors, and other small molecules throughout the matrix. Any suitable material may be used for the matrix, although the materials named herein are preferred. Example materials which we believe may be used include hydrogel type materials such as alginate or agarose. Other suitable matrix materials may include Matrigel (RTM), or extracellular matrix (ECM) material extracted from adipose tissue.

The step of introducing preadipocytes into the matrix may comprise seeding preadipocytes into a matrix precursor in a fluid form, and allowing the matrix precursor to solidify to form the matrix. The solidification may comprise polymerisation of the matrix precursor or the like.

The method may further comprise the step of isolating preadipocytes from adipose tissue for introduction into the matrix. Techniques for such isolation are known, and may include, for example, digesting adipose tissue with for example collagenase, and separating preadipocytes from other components of adipose tissue by for example filtration or centrifugation.

The method may further comprise the step of introducing one or more differentiation factors into the matrix, to cause the preadipocytes to differentiate into adipocytes. The differentiation factors may be introduced simultaneously with introduction of the preadipocytes, or previously or subsequently. The factors may be introduced into a matrix precursor in fluid form, and the precursor allowed to solidify to form the matrix. Alternatively, the factors may be introduced into the matrix by for example contacting the matrix with a liquid solution including the factors.

Suitable differentiation factors are known in the art, and may comprise, for example, those recited in U.S. Pat. No. 6,153,432, the contents of which are incorporated herein by reference. Particularly preferred differentiation factors comprise each of glucose, a cyclic AMP inducer, a glucocorticoid or glucocorticoid analogue, insulin or an insulin analogue, and a PPARγ agonist or RXR agonist. A most preferred differentiation factor is each of isobutylmethylxanthine, dexamethasone, and insulin.

The method may further comprise the step of replacing the differentiation factors in the matrix with another medium. For example, the matrix may be washed in standard growth medium (e.g. MEMα medium) after the preadipocytes have committed to differentiation.

Additional cell types may be introduced into the matrix; these may be introduced concurrently with the preadipocytes, after differentiation into adipocytes, or before introduction of the preadipocytes. Preferably the additional cell type does not include endothelial cells.

The method may comprise the step of releasing the differentiated adipocytes from the matrix. This may be achieved by for example digestion of the matrix (e.g. using collagenase if the matrix is collagen) followed by recovery of the adipocytes.

According to a further aspect of the present invention there is provided a three dimensional support matrix having preadipocytes located therein, wherein the matrix does not contain endothelial cells. The matrix may further comprise one or more differentiation factors to promote differentiation of the preadipocytes into adipocytes.

The present invention also provides a three dimensional support matrix having adipocytes located therein, the adipocytes being obtained by differentiation of preadipocytes within said matrix. Preferably also the matrix does not comprise endothelial cells.

Also provided according to the present invention is a method of investigating the response of adipocytes to stimuli, the method comprising culturing adipocytes in vitro in accordance with the methods described herein; introducing a stimulus to the adipocytes within the matrix; and determining the response of the adipocytes to the stimulus.

Suitable stimuli include drugs, drug candidates, small molecules, bioactive molecules, peptides, peptide fragments, fatty acids, nucleic acids, growth factors, differentiation factors, and the like. The step of determining the response of the adipocytes may comprise comparing the adipocytes exposed to the stimulus with adipocytes cultured in the same manner which have not been exposed to the stimulus.

The present invention also provides a method of investigating interactions between adipocytes and other cell types, the method comprising culturing adipocytes in vitro in accordance with the methods described herein; introducing another cell type into the matrix; and determining the interaction of the adipocytes with the other cell type. Alternatively, the method may comprise culturing adipocytes in vitro in accordance with the methods described herein, wherein the matrix comprises another cell type; and determining the interaction of the adipocytes with the other cell type. For either or both of these methods, the other cell type is preferably not an endothelial cell.

A still further aspect of the present invention provides a method of investigating the development of stem cells, the method comprising introducing one or more stem cells into a three dimensional support matrix having adipocytes located therein; and allowing the stem cells to grow or differentiate. The method may instead comprise culturing adipocytes in vitro in accordance with the methods described herein, wherein the matrix comprises one or more stem cells. In a yet further variation, the method may comprise introducing one or more stem cells into a three dimensional support matrix having preadipocytes located therein; allowing the preadipocytes to differentiate into adipocytes; and allowing the stem cells to grow or differentiate. Additional cell types, whether stem cells or other cells, may also be present in the matrix. This may help more closely mimic the in vivo environment; for example, osteocytes may be used to mimic bone marrow.

For any or all of the methods described herein, activity or interactions of the adipocytes may be monitored using techniques known in the art; for example, by immunostaining, detection of biomolecules within the matrix, or detection of molecules secreted from the matrix into a surrounding medium.

### BRIEF DESCRIPTION OF FIGURES

These and other aspects of the invention will now be described by way of example only with reference to the accompanying drawings, in which

FIG. 1 shows phase contrast images of 3D cultures at days 0, 7 and 14 after initiating differentiation. Differentiation is marked by the accumulation of lipid droplets within the cells.

### DETAILED DESCRIPTION OF THE INVENTION

A three-dimensional tissue culture system has been developed in which preadipocytes isolated from rats can be grown and differentiated. The mature adipocytes produced in this way show morphological and biochemical properties similar to those shown by adipocytes in vivo and ex vivo, and different from those exhibited by adipocytes grown in two-dimensional cultures. Adipocytes cultured in our 3D system maintain viability for several weeks, allowing short and long term functions to be studied.

This system provides a microenvironment more relevant to the living animal than traditional two-dimensional culture systems.

It has several areas of application, including, but not limited to:

1. It can be used to culture adipocytes alone and study their responses to environmental stimuli including bioactive molecules and therapeutic drugs;
2. It can be used to study interactions between adipocytes and other cell types;
3. It can be used to mimic microenvironments in which stem cells develop;
4. It serves to reduce the number of animals required for research and development in areas involving adipocytes.

Preadipocytes are isolated from adipose depots according to a method modified from Cabrero et al., 2001 (Diabetes. 2001 August; 50(8):1883-90). The procedure involves collagenase treatment and filtration, and yields in the order of 3-4×10⁵ cells per gram of starting tissue. The preadipocytes are seeded into a three-dimensional gel made of type I collagen in culture medium in a 24-well tissue culture plate. Under these conditions the collagen gel becomes tethered to the walls of the vessel, minimising shrinkage. When the gel sets, the preadipocytes are suspended within the 3D gel matrix. They are induced to differentiate into mature adipocytes using the differentiation medium described by Cabrero et al (2001). Once the cells are committed to differentiate, the culture medium can be changed for optimum maintenance conditions and, if required, other cell types introduced into the gel. Small cells can migrate around the gel (mature adipocytes, because of their large size, cannot) and can establish structural and functional relationships with each other and with the adipocytes.

The adipocytes, with or without other cells, can be studied in situ by immunochemistry and microscopy, or by biochemical analysis of the molecules they secrete into the culture medium. Alternatively, cells can be released from the gel by collagenase treatment, collected, and subjected to molecular analysis.

### Methods

### Preadipocyte Isolation

Animals were bred in-house and maintained in accordance with the Animals (Scientific Procedures) Act, 1986. Adult (aged 7-9 months) female Sprague Dawley rats (200-350 g) were killed by Schedule 1 methods. The left and right popliteal adipose depots were dissected out. A method described by Cabrero and co-workers (Cabrero et al., 2001) was modified to isolate and differentiate preadipocytes from adipose tissue. Method modifications included using 5 ml of type II collagenase (4 mg/ml in Hanks balanced salt solution with 10% bovine serum albumin) (Sigma-Aldrich Company Ltd, UK) for digestion for 30 minutes at 37° C. and a 200 μm cell strainer was used to remove debris and isolate mixed cells from adipose tissue including preadipocytes. The isolated cells were cultured with Minimal Essential Medium-alpha (MEM-α), without ribonucleosides, or deoxyribonucleoside and with glutaMAX-I (Gibco, Invitrogen Ltd, UK) supplemented with 5% heat-inactivated fetal bovine serum (HI-FBS) (Gibco, Invitrogen Ltd, UK) and 140 U/ml penicillin and streptomycin (P/S). 60-70% of the isolated cells are preadipocytes as judged by differentiation (assessed by Oil Red O staining) in 2D culture.

### Cell Viability After Isolation

Around 80% of the isolated cells were viable as assessed by flow cytometry or trypan blue exclusion.

### Three-Dimensional Collagen Gel Culture System

The collagen gel consisted of 1.8 mg/ml rat tail type I collagen (First Link Ltd, UK), 5% MEM×10 (Gibco, Invitrogen Ltd, UK) and 5% MEM-α (containing 5% FBS and 140 U/ml P/S) on ice. Increments of a sterile filtered 1 mol 1⁻¹ NaOH solution were added to the gel mixture until a change in colour (from yellow to deep pink) was observed indicating a change in pH. Aliquots containing 1×10⁵ viable cells were seeded in 1 ml collagen and plated in 24 well plates containing 13 mm glass coverslips (Alana Ecology, UK) at the base of the well for easy removal of the collagen gel. The gel was allowed to set for 15-20 minutes at 37° C. in a humidified incubator with 5% CO₂ and 95% air. When the gel was set, 1 ml of MEM-α medium was overlaid per well and the cultures were maintained in the incubator. Differentiation was induced as described below within three hours of seeding.

### Preadipocyte Differentiation

Differentiation was induced by the addition of MEM-α supplemented with isobutylmethylxanthine (0.5 mmol/l), dexamethasone (0.25 μmol/l) and insulin (10 μg/ml), After 48 hours, the induction medium was removed and replaced by MEM-α (containing 5% FBS and 140 U/ml P/S) supplemented with 10 μg/ml of insulin. The cultures were replenished every 2 days, replacing 50% of the medium each time. The cells were regarded as differentiated at day 14, as assessed by morphological characteristics and Oil red O staining of differentiated cells by standard methods (Bancroft, John and Stevens, 1990). The cells were viewed using a Nikon Microphot-FX microscope (Nikon UK limited, Kingston upon Thames, UK).

### Experimental Results

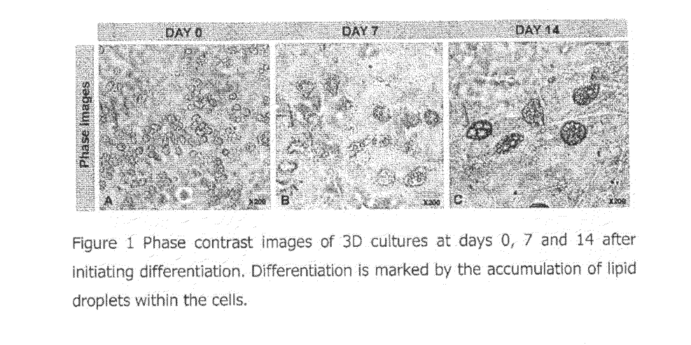

The results are shown in FIG. 1. This gives phase contrast images of 3D cultures at days 0, 7 and 14 after initiating differentiation. Differentiation is marked by the accumulation of lipid droplets within the cells. It can be seen that the protocol permits differentiation of preadipocytes to adipocytes, which appear morphologically normal within the three dimensional culture,

1. A method of culturing adipocytes in vitro, the method comprising
introducing ex vivo preadipocytes into a three dimensional support matrix; and
allowing the preadipocytes to differentiate in vitro into adipocytes within the support matrix.

2. The method of claim 1, wherein the matrix is a collagen matrix.

3. The method of claim 2 wherein the matrix is a type I collagen matrix.

4. The method of claim 1, wherein the matrix comprises a hydrogel type material.

5. The method of any preceding claim wherein the step of introducing preadipocytes into the matrix comprises seeding preadipocytes into a matrix precursor in a fluid form, and allowing the matrix precursor to solidify to form the matrix.

6. The method of any preceding claim further comprising the step of isolating preadipocytes from adipose tissue for introduction into the matrix.

7. The method of claim 6, wherein the isolation step comprises digesting adipose tissue with collagenase, and separating preadipocytes from other components of adipose tissue.

8. The method of any preceding claim further comprising the step of introducing one or more differentiation factors into the matrix, to cause the preadipocytes to differentiate into adipocytes.

9. The method of claim 8 wherein the differentiation factors are introduced simultaneously with introduction of the preadipocytes.

10. The method of claim 8 wherein the differentiation factors are introduced into a matrix precursor in fluid form, and the precursor allowed to solidify to form the matrix.

11. The method of claim 8 wherein the differentiation factors are introduced into the matrix by contacting the matrix with a liquid solution including the factors.

12. The method of any of claims 8 to 11 wherein the differentiation factors comprise each of glucose, a cyclic AMP inducer, a glucocorticoid or glucocorticoid analogue, insulin or an insulin analogue, and a PPARγ agonist or RXR agonist.

13. The method of any of claims 8 to 11 wherein the differentiation factors comprise each of isobutylmethylxanthine, dexamethasone, and insulin.

14. The method of any of claims 8 to 13 further comprising the step of replacing the differentiation factors in the matrix with another medium.

15. The method of any preceding claim wherein additional cell types may be introduced into the matrix.

16. The method of claim 15 wherein the additional cell type does not include endothelial cells.

17. The method of any preceding claim further comprising the step of releasing the differentiated adipocytes from the matrix.

18. A three dimensional support matrix having adipocytes located therein, the adipocytes being obtained by in vitro differentiation of ex vivo preadipocytes within said matrix.

19. The matrix of claim 18, wherein the matrix does not comprise endothelial cells.

20. A method of investigating the response of adipocytes to stimuli, the method comprising culturing adipocytes in vitro in accordance with the method of any of claims 1 to 17; introducing a stimulus to the adipocytes within the matrix; and determining the response of the adipocytes to the stimulus.

21. The method of claim 20 wherein the stimulus is selected from the group comprising drugs, drug candidates, small molecules, bioactive molecules, peptides, peptide fragments, fatty acids, nucleic acids, growth factors, and differentiation factors.

22. The method of claim 20 or 21 wherein the step of determining the response of the adipocytes comprises comparing the adipocytes exposed to the stimulus with adipocytes cultured in the same manner which have not been exposed to the stimulus.

23. A method of investigating interactions between adipocytes and other cell types, the method comprising culturing adipocytes in vitro in accordance with the method of any of claims 1 to 17; introducing another cell type into the matrix; and determining the interaction of the adipocytes with the other cell type.

24. A method of investigating interactions between adipocytes and other cell types, the method comprising culturing adipocytes in vitro in accordance with the method of any of claims 1 to 17, wherein the matrix comprises another cell type or types; and determining the interaction of the adipocytes with the other cell type.

25. The method of claims 23 or 24 wherein the other cell type is not an endothelial cell.

26. A method of investigating the development of stem cells, the method comprising culturing adipocytes in vitro in accordance with the method of any of claims 1 to 17, wherein the matrix comprises one or more stem cells.

27. A method of investigating the development of stem cells, the method comprising introducing one or more stem cells into a three dimensional support matrix having preadipocytes located therein; allowing the preadipocytes to differentiate into adipocytes; and allowing the stem cells to grow or differentiate.

28. The method of either of claims 26 or 27, wherein the matrix comprises another cell type or types.
